(19) United States
(12) Patent Application Publication
(10) Pub. No.: US 2025/0014748 A1
(43) Pub. Date: Jan. 9, 2025

(54) GRAPH FUSION SYSTEM FOR EMBEDDING DISPARATE DOMAIN DATA

(71) Applicant: IQVIA Inc., Parsippany, NY (US)

(72) Inventors: Tong Wu, Collegeville, PA (US); Yong Cai, Marina, CA (US); Yunlong Wang, Malvern, PA (US); Fan Zhang, Malvern, PA (US); Emily Zhao, Wayne, PA (US); Yilian Yuan, Wayne, PA (US)

(21) Appl. No.: 18/347,237

(22) Filed: Jul. 5, 2023

(51) Int. Cl. G16H 50/20; G16H 50/70

(52) U.S. Cl. CPC G16H 50/20; G16H 50/70

(57) ABSTRACT

Methods, systems, and apparatus, including computer programs encoded on computer storage media, for generating data structures from graphs. The computer accesses a graph having patient nodes representing patients and patient health data nodes representing health data for patients, the nodes being connected by edges. The computer generates subgraphs by identifying patient nodes and patient health data nodes associated with a particular healthcare provider. The computer generates, by a subgraph neural network, a healthcare provider data structure for a respective subgraph. The computer generates, by a first graph neural network, patient data structures for a respective patient graph network and health data structures for a respective health data graph network. Each healthcare provider data structure, patient graph network, health data structure, has a lower dimension than the corresponding subgraph, patient graph network, and health data graph network, respectively. The computer provides at least one of the data structures to a model.

### TECHNICAL FIELD

This specification relates to healthcare data stored in graph networks.

### BACKGROUND

Data in the healthcare industry can be derived from a variety of sources that include, patients, healthcare organizations, healthcare providers, etc. Predictive models can be used to analyze data sources from seemingly disparate sources to identify trends for applications such as responsive determination of patient triggers, alert physicians of key symptoms, detecting occurrences and predicting spread of diseases, etc.

### SUMMARY

Predictive models analyze data from multiple sources to generate predictions that can improve the practice of medicine, such as providing insights to healthcare providers to improve the quality of care for their patients. Predictive models can be deployed on platforms, software applications, and standalone on computing devices and systems to leverage multi-domain healthcare data. By processing data and generating predictions, predictive models can also improve processes adjacent and related to patient care, such as insurance claim processing for patients and drug manufacturing production targets by pharmaceutical manufacturers. Predictive models can also leverage data provided by different parties in the healthcare industry that capture patient data that includes data related to the patient, e.g., name, address, age, and patient health data, e.g., height, weight, symptoms, medication list, family medical history.

Some approaches for predictive models primarily rely on analysis based on patient data and patient health data without consideration of heterogeneous data sources, e.g., data provided in an adjacent domain to patient data and patient health data. These approaches are often limited to processing and analyzing homogenous data sources, due to the computational complexity of re-formatting and re-factoring of inputs from data sources due to different data formats. In some examples, predictive models will independently process data from heterogeneous sources without consideration of relationships between seemingly disparate, yet related data sources. For example, heterogeneous data sources can often have a complex relationship with other types of healthcare data, including patient health data, healthcare provider data, etc. that is obscured by the differences in format, type, and/or origin of the data. Some examples of heterogeneous data sources can include payer access data, healthcare provider companion data, healthcare provider digital activity data, and healthcare organization affiliation data.

The complex relationships in medical practice can be represented by a graph network that includes edges and nodes to describe relationships between patients, symptoms, prescriptions, diagnoses, and healthcare providers. Graph networks can be a helpful visual in illustrating relationships between patient data, patient healthcare data, and healthcare provider association, but often can be difficult to process as an input for a predictive model, e.g., without performing some form of encoding and/or embedding. Some approaches may include independently generating embeddings for each heterogeneous data source, but these approaches do not consider complex relationships and trends between homogenous data sources such as patient health data, and the heterogeneous data sources from adjacent medical-related domains. For example, predictive models leveraging existing embedding approaches can consume significant computational resources when generating embeddings for each type of data source, as each type of data source is processed independently. Furthermore, the independent processing of data sources often limits the fidelity of predictions generated by the predictive model,

The techniques described in this specification for generating embeddings from heterogeneous data sources include fusing the data sources, by applying graph neural networks and subgraph neural networks to a graph that represents patients and their respective health data. The patient healthcare data can include prescriptions, diagnoses, and symptoms as prescribed and/or observed by the patient's healthcare provider(s). By generating embeddings based on a graph network, the techniques described in this specification capture complex relationships between healthcare provider networks, patient networks, and patient health data subnetworks, to provide scalable and high-fidelity input embeddings to predictive models. The techniques include generating node embeddings corresponding to patient data and patient health data, and subgraph embeddings that correspond to healthcare providers and other types of disparate, heterogeneous data sources.

The embeddings are data structures with encoded values that represent the features of data, e.g., including relationships between nodes of a graph network, in a lower dimension than the data represented in the graph network. In particular, the techniques include applying machine learning techniques to graph-based networks (e.g., including subgraphs and subgraph components of the graph) to generate lower-dimensional embeddings. The lower dimensional embeddings can be more efficiently processed and scaled by a predictive model (e.g., compared to processing data from data sources), and also provide higher fidelity than embeddings that are not derived from complex and/or dense graph networks. Furthermore, the embeddings provide that predictive models have a lower bias by incorporating disparate sources and thereby avoiding weighing one type of data source more heavily than another data source. By doing so, the predictive models can generate predictions that leverage the heterogeneity of data sources provided across disparate domains of healthcare data. The techniques described in this specification provide predictive models to accurately generate predictions to inform healthcare providers and other stakeholders in the healthcare industry to treat patients.

In one general aspect, a method includes: accessing a graph including nodes connected by edges, wherein: a first subset of the nodes are patient nodes each representing a corresponding patient, wherein each patient is associated with one or more healthcare providers, and a second subset of the nodes are patient health data nodes each representing health data of a corresponding patient, wherein the health data of each patient health data node is associated with at least one of the one or more healthcare providers; generating one or more subgraphs based on the graph, including, for each subgraph: identifying the patient nodes and the patient health data nodes in the graph that are associated with a particular one of the healthcare providers, and generating the subgraph including the identified nodes; generating, by a subgraph neural network, healthcare provider data structures, each healthcare provider data structure based on a respective one of the subgraphs, wherein each healthcare provider data structure has a lower dimension than the corresponding subgraph; generating, by a first graph neural network, patient data structures, each patient data structure based on a respective patient graph network in the graph, each patient graph network including a particular one of the patient nodes of the graph and the patient health care data nodes connected to the particular patient node by edges, wherein each patient data structure has a lower dimension than the corresponding patient graph network; generating, by the first graph neural network, health data structures, each health data structure based on a respective health data graph network in the graph, each health data graph network including a particular one of the patient health data nodes of the graph and the patient nodes connected to the particular patient health data node by edges, wherein each health data structure has a lower dimension than the corresponding health data graph network; and providing to a model, at least one of (i) a patient data structure from the patient data structures or (ii) a health data structure from the health data structures or (iii) a healthcare provider data structure from the healthcare provider data structures.

Other embodiments of this and other aspects of the disclosure include corresponding systems, apparatus, and computer programs, configured to perform the actions of the methods, encoded on computer storage devices. A system of one or more computers can be configured by virtue of software, firmware, hardware, or a combination of them installed on the system that in operation cause the system to perform the actions. One or more computer programs can be so configured by virtue having instructions that, when executed by data processing apparatus, cause the apparatus to perform the actions.

The foregoing and other embodiments can each optionally include one or more of the following features, alone or in combination. For example, one embodiment includes all the following features in combination.

In some implementations, the method includes receiving patient data and patient health data from one or more data sources; determining nodes and edges for the graph based on the patient data and the patient health data; and generating at least a portion of the graph based on the determined nodes and edges.

In some implementations, the method includes wherein generating the patient data structures includes, for each patient data structure: sampling, for the particular patient node of the patient data structure, one or more neighboring nodes of the particular patient node; and encoding, for the particular patient node, data from the one or more neighboring nodes to generate the patient data structure for the particular patient node.

The method includes aggregating the data from the one or more neighboring nodes to the patient data structure for the particular patient node; and generating an objective function for the patient data structure, the objective function indicative of a likelihood of preserving the patient graph network of the graph based on the patient data structure. The method also includes wherein sampling the one or more neighboring nodes of the particular patient node includes: performing a random walk with restart from the particular patient node, wherein performing the random walk with restart includes traversing the graph from the particular patient node to a selected node in the one or more neighboring nodes, wherein the selected node is selected based on a probabilistic distribution that is sampled when the random walk is performed from the particular patient node; selecting, for each type of node in the one or more neighboring nodes, a number of top random walks from the particular patient node, wherein the type of a node indicates whether the node is a patient node or a patient health data node; and performing the number of top random walks using the one or more neighboring nodes of the particular patient node. The method includes wherein selecting the number of top random walks for each node in the graph is based on a density of the graph, the density of the graph describing a ratio of edges in the graph and a maximum number of edges contained in the graph.

In some implementations, the method includes wherein aggregating the data from the one or more neighboring nodes to the patient data structure for the particular patient node includes: aggregating the data from the one or more neighboring nodes with a same node type as the particular patient node, the type of a node indicates whether the node is a patient node or a patient health data node; and combining data from at least one of the neighboring nodes, the at least one of the neighboring nodes having a different type than a type of the particular patient node.

In some implementations, the method includes encoding the data from the one or more neighboring nodes of the particular patient node includes: pre-training the first graph neural network to generate the patient data structures based on the data from the one or more neighboring nodes; and aggregating the data from the one or more neighboring nodes of the respective patient graph networks based on a type of node, wherein the type of a node indicates whether the node is a patient node or a patient health data node.

In some implementations, the method includes the first graph neural network can be trained by performing operations including: generating the patient data structures and the health data structures based on respective graph networks from the accessed graph; combining the patient data structures and the health data structures to generate a reconstructed graph; comparing the reconstructed graph based on the combined patient data structures and health data structures to the accessed graph; and based on the comparison of the reconstructed graph and the accessed graph, updating one or more parameters of the first graph neural network.

In some implementations, the method includes the subgraph neural network can be trained by performing operations including: generating the healthcare provider data structures based on the respective subgraphs from the accessed graph; combining the healthcare provider data structures to generate a reconstructed graph; comparing the reconstructed graph based on the healthcare provider data structures to the accessed graph; and based on the comparison of the reconstructed graph and the accessed graph, updating one or more parameters of the subgraph neural network.

In some implementations, the method includes wherein the first graph neural network is a heterogeneous neural network that includes a long short-term memory network configured to aggregate data from the nodes based on an order of the nodes.

In some implementations, the method includes wherein the health data of a patient includes at least one of (i) a diagnosis, (ii) a prescription, or (iii) a symptom, for the patient.

In some implementations, the method includes generating a second graph representing a healthcare provider network based on the healthcare provider data structures and external healthcare data, the second graph including healthcare provider nodes each representing a respective healthcare provider, the healthcare provider nodes connected by edges. The method includes wherein the external healthcare data includes data related to one or more of (i) healthcare organizations, (ii) payer access, (iii) physician early adoption, or (iv) healthcare provider marketing. The method includes generating, by a second graph neural network, second healthcare provider data structures, each second healthcare provider data structure based on a respective healthcare provider graph network in the second graph, each healthcare provider graph network including a particular one of the healthcare provider nodes of the second graph and nodes connected to the particular healthcare provider node by edges, wherein each second healthcare provider data structure has a lower dimension than the corresponding healthcare provider graph network. The method includes generating, by a second graph neural network, external healthcare data structures, each external healthcare data structure based on a respective healthcare provider graph network in the second graph and the external healthcare data, each healthcare provider graph network including a particular one of the healthcare provider nodes of the second graph and nodes connected to the particular healthcare provider node by edges, wherein each external healthcare data structure has a lower dimension than the corresponding external healthcare data.

In some implementations, the method includes wherein the first graph neural network and the second graph neural network are the same neural network.

In some implementations, the method includes wherein generating a particular healthcare provider data structure includes: determining a set of internal nodes and a set of border nodes within the subgraph for the health care provider data structure, each internal node connected by edges to two or more nodes in the subgraph and each border node corresponding to a node of the subgraph within a radial distance of the internal nodes within the subgraph; defining a set of characteristics for each subgraph for the health care provider data structure based on the set of internal nodes and the set of border nodes; generating a set of anchor patches the graph, each anchor patch representing a random sample of the nodes and the edges of the graph; providing one or more messages from the set of anchor patches to the set of internal nodes, the set of border nodes, and the edges of the subgraph, the one or more messages encoding properties of at least one anchor patch from the set of anchor patches onto at least one of (i) the set of internal nodes, (ii) the set of border nodes, or (iii) the edges, of the subgraph; and generating, based on the set of characteristics and the one or more messages for the subgraph, the particular healthcare provider data structure.

In some implementations, the method includes wherein defining the set of characteristics for a particular subgraph includes: computing a border distance from each node in the set of border nodes of the subgraph to neighboring nodes of the graph, wherein the subgraph does not include neighboring nodes of the graph and the neighboring nodes are adjacent to at least one node in the set of border nodes; computing, for each node in the subgraph, an internal distances from a respective node in the subgraph to other nodes in the subgraph; identifying, for each node in the subgraph, a set of subgraph neighboring nodes, wherein the set of subgraph neighboring nodes for a respective node includes nodes of the subgraph within a radial distance of the respective node; and determining a structure of the subgraph based on the set of subgraph neighboring nodes for each node in the subgraph, the structure of the subgraph including a degree of connectivity for at least one (i) the nodes in the subgraph, or (ii) the edges of the subgraph connecting to the set of internal nodes of the subgraph.

In some implementations, the method includes wherein generating the particular healthcare provider data structure includes: sampling the random sample of nodes and edges from the set of anchor patches based on the one or more messages for the subgraph; encoding each anchor patch in the set of anchor patches to a lower dimension than a dimension of the corresponding anchor patch; and estimating a similarity of each anchor patch in the set of anchor patches to the respective subgraph based on the set of characteristics for subgraph.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIGS. 1A-1C are block diagrams that illustrate an example of a graph fusion system for generating embeddings of a graph representing healthcare data from disparate data sources.

FIG. 2 is a block diagram that illustrates an example of a graph neural network of the graph fusion system, the graph neural network configured to generate node embeddings.

FIG. 3 is a block diagram that illustrates an example of a subgraph neural network of the graph fusion system, the subgraph neural network configured to generate subgraph embeddings.

FIG. 4 is a flow diagram that illustrates an example of a process for generating embeddings of a graph representing healthcare data from disparate data sources using a graph fusion system.

FIG. 5 is a flow diagram that illustrates an example of a process for training a graph neural network of a graph fusion system to generate node embeddings.

FIG. 6 is a flow diagram that illustrates an example of a process for training a subgraph neural network of a graph fusion system to generate subgraph embeddings.

FIG. 7 shows a block diagram of a computing system.

Like reference numbers and designations in the various drawings indicate like elements. The components shown here, their connections and relationships, and their functions, are meant to be examples only, and are not meant to limit the implementations described and/or claimed in this document.

### DETAILED DESCRIPTION

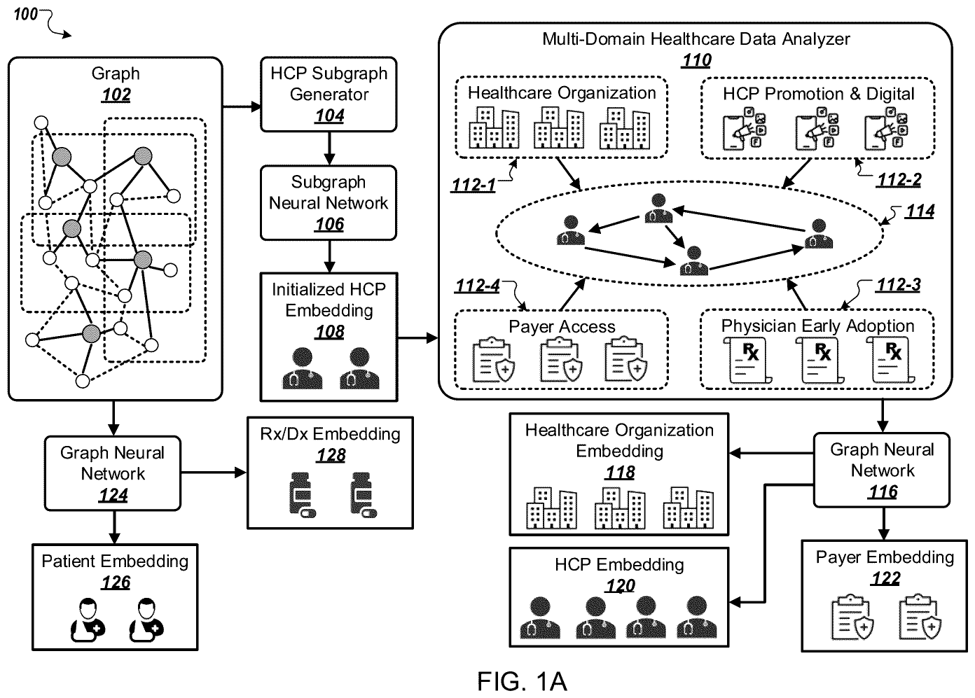

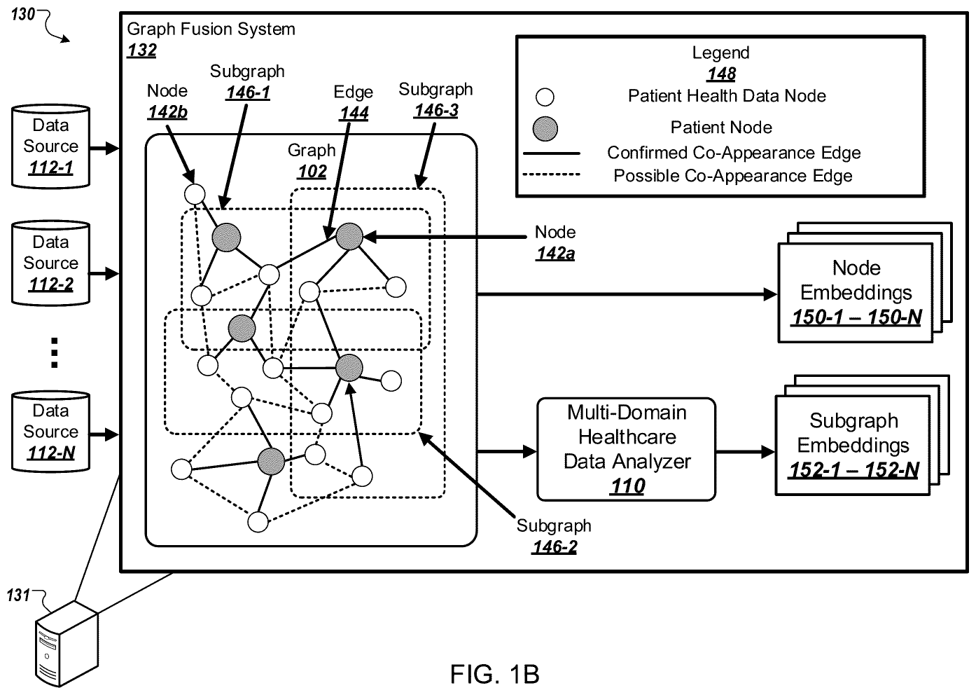

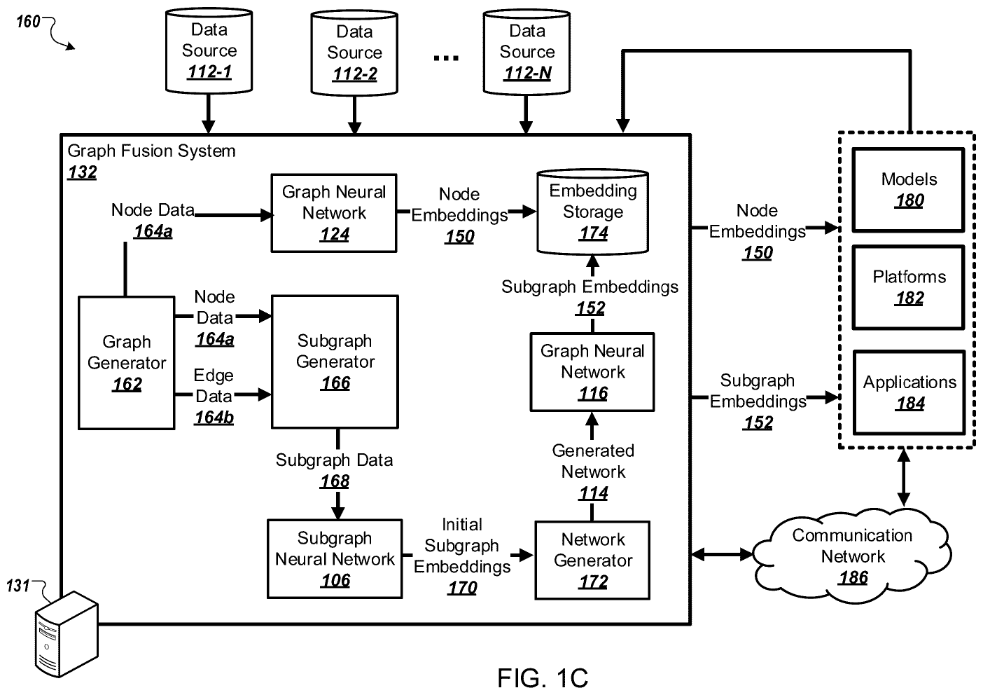

FIG. 1A is a block diagram that illustrates an example of a system 100 for generating embeddings of a graph representing healthcare data from disparate data sources. System 100 can be a computing system, device, or collection of devices configured to process input data, such as patient data and patient health data represented by the graph 102. Patient data describes a patient's identification information such as name, age, address, etc. and patient health data indicates a health information, such as symptoms, diagnoses, and prescriptions.

The system 100 generates embeddings of the data represented in the graph 102, which are lower dimensional representations of the features in the data of the graph 102, e.g., the graph 102 generated from heterogeneous and/or disparate data sources. By generating embeddings of the data in the graph 102, the system 100 can provide that relationships between patient data, patient health data, and other types of data (e.g., data related to the healthcare providers of the patient) are captured in the embedding. The embedding can be a vector-based and/or tensor-based data structure, in which each element of the data structure can be a value, e.g., floating point, double type. In some implementations, the values of the data structure can be limited to a range of values, e.g., ranging from −1 to 1, 0 to 1, or any appropriate bounds. The embedding can be provided as data input for a predictive model, e.g., thereby enabling the predictive model to predict trends across disparate data sources and types.

The graph 102 includes nodes and edges, in which each node represents an object, a place, a person, etc., while edges represent relationships between two or more nodes. The graph 102 includes nodes representing patients (referred to as patient nodes) and patient health data, e.g., symptoms, diagnoses, and prescriptions (referred to as patient health data nodes). As shown in FIG. 1A, nodes of a different type can be illustrated by a different color, e.g., a gray circle for a patient node and a white circle for a patient health data node. An edge connecting a patient node to a patient health data node indicates that the patient health data node is applicable to the connected patient node, e.g., a patient experienced a symptom, received a diagnosis, or was prescribed a medication. Furthermore, nodes of the graph 102 are associated with a healthcare provider. Patient nodes associated with a particular healthcare provider indicate that the patients of the patient nodes received care from the particular healthcare provider. Patient health data nodes associated to a particular healthcare provider indicate patient health data such as prescriptions, symptoms, and diagnoses that originated from the particular healthcare provider.

Furthermore, the graph 102 can include subgraphs that include groupings of a subset of the nodes and edges of the graph 102, e.g., a portion of the graph. For instance, the nodes and edges that constitutes a subgraph share a common characteristic or correspond to a common label, e.g., a description describing the relationship between two or more of the nodes of the subgraph. In an example, the nodes of a subgraph are all associated with a particular healthcare provider, as discussed in the next paragraph. In some implementations, the subgraph is a randomly selected grouping of connected nodes and the respective edges that connect one node to another node. For example, a subgraph of the graph 102 can include the patient nodes, e.g., nodes representing patients, corresponding to a particular health care provider, thereby indicating that the patients represented by the patient nodes were treated by the particular healthcare provider. The subgraph also includes the patient health data nodes, e.g., nodes representing patient health data, corresponding to the same particular healthcare provider, and indicate that the particular healthcare provider observed symptoms, prescribed medication, and/or diagnosed medical conditions, for patients of the patient nodes in the subgraph. Additionally, the subgraph includes the edges of the subgraph that connect the patient nodes and patient health data nodes of the subgraph.

The system 100 processes the graph 102 using a healthcare provider (HCP) subgraph generator 104 to generate subgraphs, each subgraph corresponding to a particular healthcare provider. The HCP subgraph generator 104 identifies the healthcare providers represented in the graph 102, and identifies respective sets of patient nodes and patient health data nodes in the graph 102 associated with each identified healthcare provider. The identified set of nodes for each healthcare provider constitute a subgraph for that healthcare provider. In some examples, any patient nodes and patient health data nodes associated with a given healthcare provider are included in the subgraph for that healthcare provider. In some examples, a patient node connected to a patient health data node by an edge is included in a subgraph for a respective healthcare provider if both the patient node and the patient health data node are associated with the respective healthcare provider, e.g., the healthcare provider prescribed medication (represented by the associated patient health data node) to the patient (represented by the associated patient data node).

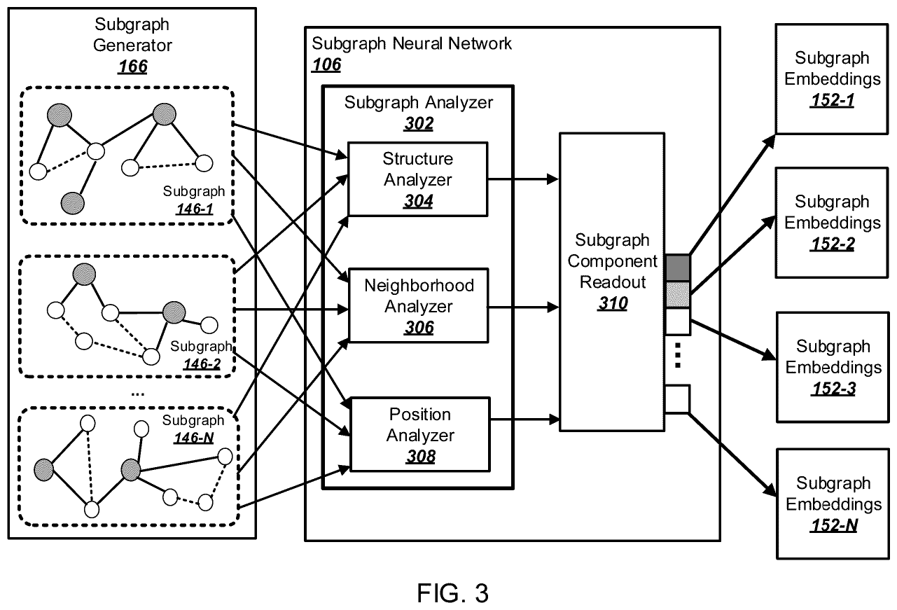

The subgraphs generated by the HCP subgraph generator 104 are processed by a subgraph neural network 106 to generate an initialized HCP embedding 108 for each subgraph, e.g., a data structure with initial values. Each initialized HCP embedding 108 (e.g., subgraph embedding) corresponds to a subgraph for a respective healthcare provider, in which the initialized HCP embedding is a lower dimensional representation of the data (e.g., from the nodes and edges) from the subgraph for the respective healthcare provider. The subgraph neural network 106 can be a machine learning model trained to generate embeddings of subgraphs, e.g., to embed data of the healthcare provider subgraph into values of a data structure. Further details of the subgraph neural network 106 are described in reference to FIG. 3 below.

The initialized HCP embedding 108 is analyzed by a multi-domain healthcare data analyzer 110, which generates a physician peer network 114 using data sources 112-1-112-4 (collectively referred to as “data sources 112”). The physician peer network 114, also referred to as a “generated network,” includes data corresponding to multiple healthcare providers, e.g., multiple subgraph embeddings. The physician peer network 114 is a graph network that includes nodes and edges representing data from the data sources 112, e.g., in addition to nodes representing healthcare providers and edges representing relationships between the healthcare providers. The multi-domain healthcare data analyzer 110 can leverage different types of data represented by the data sources 112 to build the physician peer network 114, thereby incorporating additional insights, trends, and/or patterns. Although illustrated as four disparate data sources, any suitable number of data sources (e.g., including non-disparate sources that are similar in type/format) can be utilized for generating the physician peer network 114.

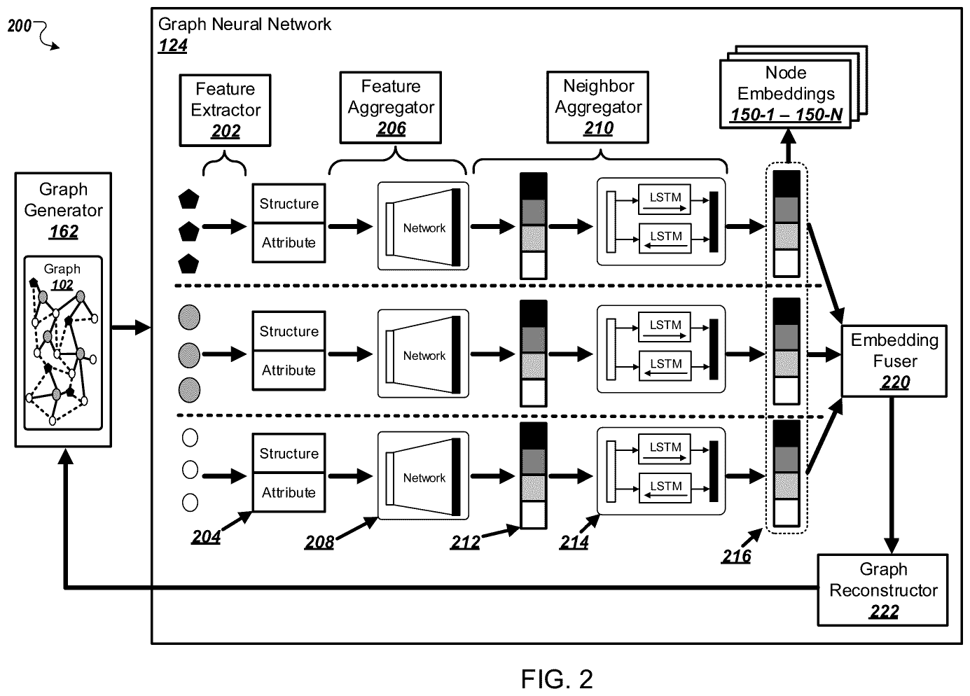

The system 100 applies a graph neural network 116 to generate embeddings 118, 120, 122 of the physician peer network 114, e.g., incorporating. The graph neural network 116 can be a machine learning model trained to generate embeddings of graphs, e.g., to embed data of the physician peer network 114 into values of a data structure. Further details of the graph neural network 116 are described in reference to FIG. 2 below. The embeddings 118, 120, 122 are lower dimensional representations of the data represented in the physician peer network 114, which incorporates some or all of the data included in data sources 112. The physician peer network 114 incorporates data from multiple sources (e.g., heterogeneous, disparate) as a graph network with nodes representing healthcare providers and edges representing relationships between two or more healthcare providers. As a first example, the data source 112-1 indicates data related to healthcare organizations that are associated with the healthcare providers in the physician peer network 114. The data from data source 112-1 can indicate known associations between medical professionals in the generated network and medical organizations, e.g., such as a physician affiliated with multiple hospitals, primary care offices. Data from healthcare organizations can help describe relationships between multiple healthcare providers in the physician peer network 114 across multiple subgraphs, thereby identifying which of the healthcare providers are associated with the same healthcare organization. As a second example, the data source 112-2 indicates data related healthcare provider promotions and digital marketing, which can include advertisements and other marketing materials tailored to healthcare providers. As a third example, the data source 112-3 indicates data related to physician early adoption, which can indicate a time in which a healthcare provider of the physician peer network 114 adopts medications, drugs, types of medical devices, etc. as a form of treating patients. As a fourth example, the data source 112-4 indicates data related to payer access that describe insurance coverage and policies accepted by the healthcare providers in the physician peer network.

The graph neural network 116 provides that the generated embeddings capture relationships between the data sources that are represented by nodes and edges of the physician peer network 114. Multiple types of embeddings are generated by the graph neural network 116, each type of embedding representing a particular set of features. The graph neural network 116 provides that each type of embedding combines and encodes features across multiple data sources 112 by consolidating all types and kinds of data into a data structure for a particular type of embedding. The graph neural network 116 generates healthcare organization embedding 118, HCP embedding 120, and payer embedding 122 as lower dimensional representations of node and edge data in the physician peer network 114.

The healthcare organization embedding 118 is an encoded representation of feature data from the physician peer network 114, in which the feature data can be any data from the data sources 112, e.g., beyond data related to healthcare organizations from data source 112-1. In some cases, features represented in the physician peer network 114 is sparse in a data source, e.g., a data source includes sparsely populated values. The healthcare organization embedding 118 represents features for healthcare organization data across all data sources 112, e.g., by encoding data from nodes and edges from the physician peer network 114. The healthcare organization embedding 118 describes some or all of the healthcare providers in the physician peer network 114 that are associated with the same healthcare organization(s). The values for the healthcare organization embedding 118 also includes are encoded values representing features from other data sources 112-2-112-4, e.g., by combining and encoding features in the nodes and relationships indicated by edges, of the physician peer network 114.

The HCP embedding 120 is an encoded representation of feature data from the physician peer network 114, in which each HCP embedding 120 corresponds to a healthcare provider in the physician peer network 114. In some implementations, generating the HCP embedding 120 includes updating the values in the initialized HCP embedding 108 to capture the features represented in nodes and edges of the physician peer network 114. In some implementations, the HCP embedding 120 can be a new data structure of a different type and/or size than the initialized HCP embedding 108. As an example, the graph neural network 116 generates a payer embedding 122 as a lower representation of the payer access data, e.g., coverage rates, policies, health insurance tiers, across some or all of the healthcare providers in the physician peer network 114.

In addition to the multi-domain healthcare data analyzer 110 identifying patterns between healthcare providers through data sources 112, the system 100 applies a graph neural network 124 to graph 102 to generate patient embedding 126 and Rx/Dx embedding 128. Similar to the graph neural network 116, the graph neural network 124 generates embeddings based on the patient nodes, patient health data nodes, and edges, of the graph 102. The patient embedding 126 is a data structure with values representing features of patient data, e.g., an embedding for each patient node in the graph 102 that embeds features of the data related to the patient. The patient embedding 126 includes encoded values of feature data for a patient represented in the graph 102, e.g., a particular patient node. In other words, the patient embedding 126 captures relationships across healthcare providers and patient health data nodes (e.g., by connected edges) in the graph 102 into a data structure for the patient. Similarly, the Rx/Dx embedding 128 is a data structure with values representing features of patient health data, e.g., an embedding for each patient health data node in the graph 102 that embeds features of the data related to the patient health data. The Rx/Dx embedding 128 includes encoded values of feature data for a patient health data node, e.g., prescription medication, symptom, diagnosis. The Rx/Dx embedding 128 captures relationships across patient nodes (e.g., by connected edges) and healthcare providers in the graph 102 into a data structure for the patient health data node. Further details of the graph neural network 124 are described in reference to FIG. 2 below.

The embeddings generated by graph neural networks 116, 124, and subgraph neural network 106 of the system 100 can be provided as an input for analytic and/or predictive processing. By generating a lower dimensional representation of graph network such as graph 102 and physician peer network 114, the system 100 provides data structures of encoded values that are rich (e.g., compared to the data provided by data sources 112-1-112-N) and dense (e.g., compared to node and edge representations in graph 102 and physician peer network 114). As described in this specification, the richness of the data structure indicates that features from multiple types, formats, and sources across data sources (e.g., including heterogeneous and sparse data sources) are combined into an embedding. The density of the data structure indicates that features across data sources can be consolidated into fewer instances of the data structure, e.g., compared to the data from the data sources 112. In some implementations, some or all of the embeddings are provided for input for further processing, e.g., by a model, platform, software application. In some implementations, the initialized HCP embedding 108 is outputted as a subgraph embedding for further processing. Further description of example models, platforms, and applications are described in reference to FIG. 1B below.

FIG. 1B is a block diagram 130 that illustrates an example of the system 100 in the form of a graph fusion system 132 configured to generate node embeddings 150-1-150-N (“collectively referred to as “node embeddings 150”) and subgraph embeddings 152-1-152-N (collectively referred to as “subgraph embeddings 152”). Examples of node embeddings 150 include patient embedding 126 and Rx/Dx embedding 128 as described in reference to FIG. 1A above. Examples of subgraph embeddings 152 include initialized HCP embedding 108, healthcare organization embedding 118, HCP embedding 120, and payer embedding 122, also described in reference to FIG. 1A above.

As illustrated, the graph fusion system 132 is deployed on a computing device 131, but can deployed on other types of computing devices, systems, platforms, etc. The graph fusion system 132 accesses data sources 112-1-112-N (collectively referred to as “data sources 112”) to generate the graph 102. In some implementations, the data sources 112 include node data and edge data related to graph networks and subgraph networks, which are accessed by the graph fusion system 132 to generate embeddings. As described in reference to FIG. 1A, the data sources 112 can include data related to and/or received from healthcare organizations, insurance providers, promotional materials and advertisements, etc. In some implementations, the data sources 112 also include patient data and patient health data.

The graph 102 includes subgraphs 146-1-146-3, each subgraph including nodes and edges that are associated with a respective healthcare provider. For example, the encircled nodes and edges indicated by the dashed line for subgraph 146-1 are associated to a first healthcare provider, while the respective nodes and edges encircled by subgraphs 146-2 and subgraph 146-3 are associated to a second and third healthcare provider, respectively. Although three subgraphs are illustrated in the graph 102, any number of subgraphs can be determined from the graph 102. In some implementations, some or all of the components of the graph 102 such as nodes and edges are updated (e.g., continually, periodically) by providing data from data sources 112.

Referring to the legend 148, a gray circle illustrated in the graph 102 indicates a patient node, e.g., node 142a representing a particular patient that is associated with (e.g., observed and/or being treated by) the healthcare provider in the respective subgraph of the healthcare provider. Patient data nodes can indicate patient data such as name, age, address, etc. A white circle illustrated in the graph 102 indicates a patient health data node, e.g., node 142b representing patient health data that is associated with (e.g., prescribed and/or determined by) the healthcare provider in the respective subgraph of the healthcare provider. Patient health data nodes can indicate patient health data such as symptoms, diagnoses, and prescriptions for a patient, by being connected to a patient node.

Each patient node in the graph 102 can be connected to one or more patient health data nodes, indicating multiple types and/or instances of patient health data for the patient corresponding to the patient node. Referring to legend 148, an illustration of a solid black line between a patient node and a patient health data node in the graph 102 describes an edge with a confirmed co-appearance (e.g., “confirmed co-appearance edge”) between the patient node and the patient health data node that is connected by the edge. For example, a confirmed co-appearance of a patient health data node and a patient node indicates an established relationship between the patient health data node and the patient node, e.g., the patient was prescribed medication described by the patient health data node. An illustration of a dashed black line between patient health data nodes in the graph 102 describes an edge indicating a possible co-appearance (e.g., “possible co-appearance edge”) between the two connected patient health data nodes. A possible co-appearance edge between two patient health data nodes indicates that the two instances of patient health data, e.g., diagnoses, medical procedures, symptoms, prescriptions, occur in a medical record for one or more patients. The co-occurrence can occur within a time window, e.g., a number of days, weeks, months. The possible co-appearance edge between two patient health nodes describes a likely relationship between the patient health data nodes. For example, a procedure such as a biopsy for a patient is represented by a first patient health data node, while a corresponding diagnosis based on the biopsy results is represented by a second patient health data node, e.g., a diagnosis for chronic kidney disease.

In some implementations, the graph 102 is a directed graph, in which an edge of the graph includes an arrow indicating directionality between two connected nodes, e.g., a patient node and a patient health data node. For example, an edge from a patient node can have an arrow going into a patient health data node to indicate that a symptom is self-observed, a diagnosis is self-diagnosed, and/or that a medication is self-prescribed, e.g., over-the-counter medication. In contrast, an edge starting from a patient health data with an arrow going into a patient node can indicate that a healthcare provider observed the symptom, performed the diagnosis, prescribed the medication, etc. In some implementations, the graph 102 is a weighted graph (e.g., with one or more edges of the graph 102 having an associated weight). The weight of an edge can be a value describing a relationship between two connected nodes, e.g., a quantitative characteristic of the relationship.

By processing and analyzing nodes and edges of the subgraph, machine learning techniques performed by neural networks are applied by the graph fusion system 132 to generate node embeddings 150 and subgraph embeddings 152. In the case of subgraph embeddings, the graph fusion system 132 includes the multi-domain healthcare data analyzer 110 to incorporate data from other types of sources, e.g., data sources 112-1-112-4. The machine learning techniques described in this specification provide that the node embeddings 150 and subgraph embeddings 152 can efficiently capture trends, patterns, and other types of relationships from data sources 112 that are disparate, e.g., different in source/origin, type, format, shape, size, in a lower dimension than the data represented by graph 102.

FIG. 1C is a block diagram 160 that illustrates an example of the graph fusion system 132 that includes the graph neural network 124 and the subgraph neural network 106 to generate node embeddings 150 and subgraph embeddings 152 of the graph 102, respectively. In particular, the block diagram 160 illustrates the graph fusion system 132 providing node embeddings 150 and subgraph embeddings 152 to models 180, platforms 182, and applications 184, e.g., by a communication network 186. The models 180, platforms 182, and applications 184 can be collectively referred to as “embedding applications,” which perform different types of inference, classification, and regression tasks using the embeddings as inputs. Examples of communication network 186 can include Bluetooth, Wi-Fi, the Internet, etc. In some implementations, the graph fusion system 132 is coupled to some or all of the embedding applications, by a wired connection, wireless connection, etc. Embedding applications can also include different types of downstream tasks such as healthcare provider targeting, intensive care unit readmission prediction, and disease detection prediction.

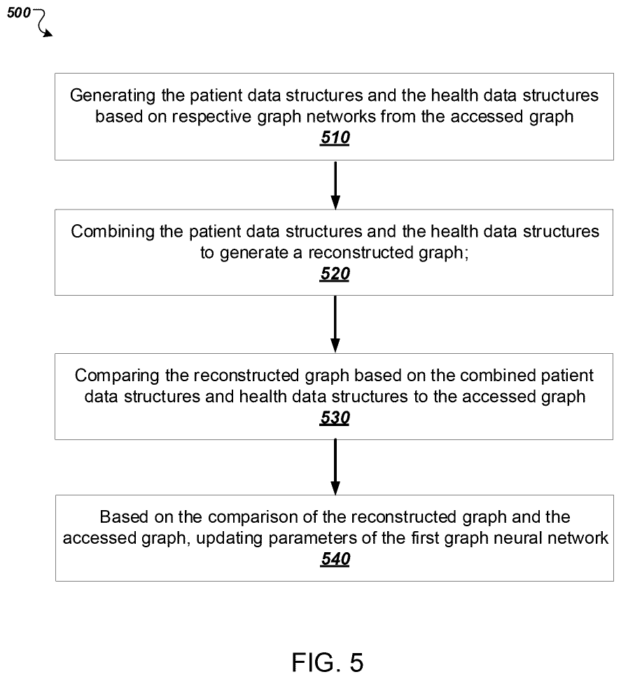

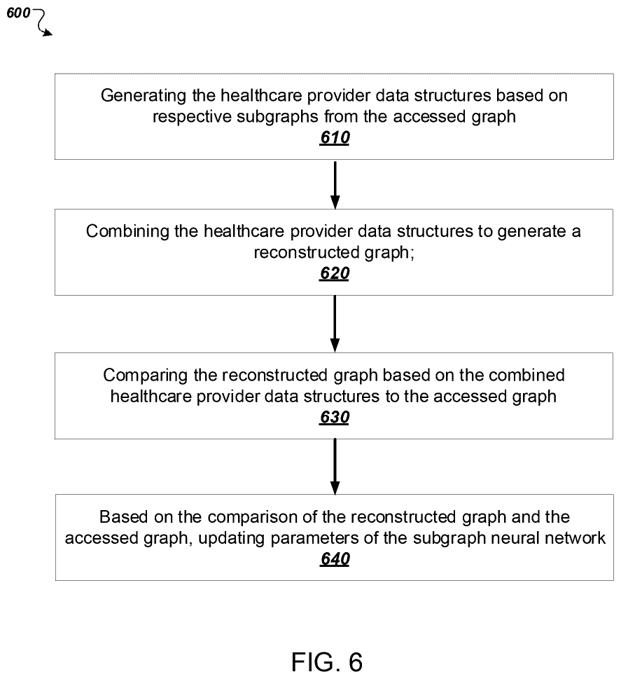

In some implementations, the models 180 include predictive models deployed on computing devices and/or systems configured to generate predictions related to future trends in patient data and patient healthcare data from the data sources 112. An example of the platforms 182 includes a computing platform configured to perform different types of data processing techniques, e.g., generating alerts related to anomalies in healthcare data, identifying patterns in types of prescriptions being provided to patients by healthcare providers. The applications 184 can include software applications, application programming interfaces, and other types of software that can be performed on computing devices, mobile devices, etc. The graph fusion system 132 includes a feedback loop with embedding applications such as models 180, platforms 182, and/or applications 184 for training (described in reference to FIGS. 5 and 6 below.)

By providing embeddings of nodes and subgraphs of the graph 102 to the embedding applications, the graph fusion system 132 provides higher fidelity data from multiple capture sources than the data that would be processed otherwise by the embedding applications. In other words, the embeddings provide a lower dimensional, standardized representation of the graph (e.g., generated from data sources), in which the embeddings can be directly used by an embedding application without adjusting the mechanism for the embedding application. For example, adjusting the embedding application can include re-structuring components of models, revising substantial lines of code in software applications, etc. This is because the embeddings can be data structures that are vector-based or tensor-based, with encoded values representing features of the data sources without needing to explicitly describe the value of the feature. The encoded values in data structures (e.g., node embeddings 150 and subgraph embeddings 152) enable sparse data to be semantically structured in a way that clusters the data together, despite the data coming from disparate sources. Reducing sparsity in data inputs for embedding applications provide that models, platforms, and applications spend less time accessing empty portions of memory to reach sparse areas of data sources, by embedding the data into a smaller feature representation (e.g., dimensionality) than the feature representation in the original data sources.

The process of embedding data is also learnable, e.g., by graph neural networks 116, 124, and subgraph neural network 106, and therefore can be trained to embed disparate sources based on semantic language identifiers, e.g., particular terms and forms of data used in the healthcare industry. Embedding data into data structures also provides standardization across data sources and reduces complexity of data inputs, thereby decreasing computational complexity for embedding applications. Therefore, embeddings can be re-used across models, platforms, applications, etc. without needing to be updated, which can further increase the utility of embeddings as data inputs. In particular, node embeddings 150 and subgraph embeddings 152 capture relationships described in graph networks such as graph 102, but also reduce the complexity from a discrete representation (e.g., a graph network that includes discrete objects including nodes and edges) to a continuous representation (e.g., vectors and/or tensors that can be processed by vector-based and/or tensor-based computing applications).

Referring to FIG. 1C, the graph fusion system includes graph generator 162 configured to generate nodes and edges for the graph 102, based on the data provided by data sources 112. In some implementations, the graph generator 162 receives streams and/or portions of data from data sources 112 to update some or all of the graph 102. The graph generator 162 provides node data 164a to graph neural network 124, which is configured to generate node embeddings 150 based on the node data 150. The graph generator 162 also provides node data 164a and edge data 164b to the subgraph generator 166, which is configured to generate subgraph data 168. The subgraph data 168 includes generated subgraphs based on the node data 164a and edge data 164b, which can include matrices, vectors, tables, and other numerical representations of graph components. The node data 164a includes data represented by patient nodes and patient health data nodes, while the edge data 164b include data indicating relationships (e.g., connections) between patient nodes and patient health data nodes, as described in reference to FIGS. 1A and 1B above.

The subgraph data 168 are provided to the subgraph neural network 106, which is trained to generate initial subgraph embeddings 170—each embedding representing a subgraph corresponding to a healthcare provider. The initial subgraph embeddings 170 are data structures with an initial set of values as element entries, e.g., elements in an array. A network generator 172 is configured to generate a generated network 114 of all of the healthcare providers through the subgraphs representing healthcare provider subnetworks. The generated network 114 is also referred to as the physician peer network 114, described in reference to FIG. 1A above. Additional data from data sources 112 can be provided to graph fusion system 132, in which the additional data includes other types and/or formats of data. Based on the additional data, the graph neural network 116 processes the generated network 114 to generate subgraph embeddings 152. One or more of the node embeddings 150 and/or the subgraph embeddings 152 can be stored in embedding storage 174, although the embeddings can be provided, e.g., transmitted, requested by, the embedding applications.

In some implementations, the graph fusion system 132 generates the node embeddings 150 and the subgraph embeddings 152 simultaneously, although the embedding process can be performed sequentially, e.g., generating node embeddings then generating subgraph embeddings or vice-versa.

FIG. 2 is a block diagram 200 that illustrates an example of the graph neural network 124 configured to generate node embeddings 150. The block diagram 200 illustrates the graph generator 162 that includes the graph 102, which can be generated, accessed, updated, etc. The graph generator 162 provides the graph 102 to the graph neural network 124, which identifies different types of nodes, e.g., patient nodes and patient health data nodes, in the graph 102. As illustrated in FIG. 2, nodes in the graph 102 are illustrated by black hexagons, gray circles, and white circles, each shape and color corresponding to a different type of node, e.g., patient health data, patient data.

The graph neural network 124 performs and/or applies different processes and techniques to generate node embeddings 150, thereby learning to encode data from a particular type to a node embedding, e.g., based on maximizing or minimizing objective functions. The graph neural network 124 performs these techniques on each type of node for all nodes that share the same type. In this specification, application of machine learning techniques can be performed on sets of nodes, as well as subgraphs, e.g., sets of nodes and corresponding edges. Furthermore, the graph neural network 124 can generate embeddings through simultaneous processing of different groups of nodes corresponding to different node types, although processing of node groupings can also be performed sequentially, out-of-order, etc.

The graph neural network 124 performs feature extraction for a type of node through the feature extractor 202, e.g., configured to extract features of nodes. The feature extractor 202 identifies features 204 for a node type, which includes structure and attribute for each node type. In some implementations, the feature extractor 202 includes a bag-of-words model to extract features from the nodes.

The structural features for a node can include different data types, e.g., linked lists, arrays, hash tables, for nodes in graph networks. As an example, patient nodes includes a string of characters indicating the name, location, age, etc., while patient health data nodes indicate pharmaceutical names for patient health data nodes, e.g., medications. Patient health data nodes indicating a symptom for a patient can include structure features indicating a plain language name for the symptom, but can also include medical terminology for the symptom, e.g., as provided by a healthcare provider. In some implementations, the structural features also include the size of the data represented by the node.

Nodes also include feature data describing attributes of the node, e.g., related to the data from data sources represented by the node. The attribute of nodes can include properties of the node, such as degree of the node, depth of the node, height of the node, etc. For example, the degree of the node indicates a number of edges that connect to the node, e.g., a node with four edges has a degree of four. Node attributes can describe how connected one node is compared to another node, e.g., a patient node with a greater degree than neighboring patient nodes indicates a greater number of patient health data nodes associated with the patient, than patients corresponding to the neighboring patient nodes.

In some implementations, the feature extractor 202 identifies structural features and attributes of nodes by sampling heterogeneous neighbors of the node. As an example, node 142a (e.g., referring to FIG. 1B) has a particular node type indicated by the color, e.g., an illustration of a gray circle for the node 142a indicates that the node is a patient node in the graph 102. The feature extractor 202 selects neighboring nodes of the node 142a with different node types, such as patient health data nodes that are connected to node 142a. The selected neighboring nodes are sampled by performing a random walk with restart, which can include traversing e.g., a walk through multiple nodes, the graph 102 by starting at the node 142a and sampling data from a node from the selected neighboring nodes at random. The random walk can include a probability of restarting the walk, indicating a likelihood that traversing to a next node in a sequence of nodes will cause the walk to return to the first node in the sequence, e.g., the node 142a, thereby resulting in a restart.

In some implementations, the probability of restart is a sampled value from a uniform probability distribution, although any type of probability distribution can be used. The probability of restart is associated to a vicinity of the node, e.g., an indication of how far in the graph the walk can be performed, and thereby related to the depth of the node when sampling data from nodes in a walk. For each node type, a top number of random walks is performed by the feature extractor 202 to identify the traversed nodes in the random walks as the heterogeneous neighbors of a particular node. By performing random walks with restart, the graph neural network is able to establish relationships between two or more nodes of different types and incorporate the disparity between the sources of data that the nodes in the random walk represent.

For each type of node, the graph neural network 124 aggregates the structural features and attribute features through the feature aggregator 206, which is configured to encode contents of the heterogeneous neighboring nodes using network 208 to generate encoded content 212. The feature aggregator 206 applies network 208 to each set of nodes corresponding to a particular node type, to encode interactions between features with different node types to generate an initial encoding for each node. In some implementations, the network 208 can be a fully-connected network configured to generate encodings of data contents of heterogeneous neighboring nodes, e.g., encoded content 212.

The network 208 encodes the contents of each node in the heterogeneous neighboring nodes to reduce the dimensionality of the data and pre-train embeddings, e.g., prior to generating a node embedding 150 for a type of node. For example, the encoded content 212 for a node type captures features of similar nodes (e.g., sharing the same node type) as an initial data structure to be aggregated. Encoding the contents of nodes can include any number of encoding techniques, including ordinal encoding, target encoding, etc. to translate feature data for the nodes into numerical values for embeddings. Furthermore, as the contents of each node type are encoding into individual embeddings, the contents of nodes with the same type are aggregated, e.g., consolidated, combined, into a single embedding that includes the heterogeneous contents of heterogeneous neighboring nodes for a particular type of node.

In some implementations, the network 208 applied to each type of node, e.g., each set of nodes sharing the same node type, is trained to generate encoded contents that are tailored to the particular node type. In some implementations, the network 208 is a multi-layer perceptron network (MLP) with multiple feed-forward layers, e.g., fully connected layers.

In some implementations, each network 208 is a distinct MLP configured to generate encoded content 212 from the heterogeneous neighboring nodes. Each layer of the MLP can be configured to apply an affine transformation to the input to the layer, e.g., multiplying an input vector to the layer by a weight matrix of the layer to encode the contents of the node. Optionally, one or more of the fully-connected layers can apply a non-linear activation function to the output of the affine transformation to generate the output of the layer, e.g., encoded node contents. Some examples of non-linear activation functions include ReLU, logistic, hyperbolic tangent, etc.

The encoded content 212 for each node type is aggregated by neighbor aggregator 210, which is configured to apply a long short-term memory (LSTM) network 214 to generate aggregated neighbor embeddings 216. The LSTM network 214 for each type of network is illustrated in FIG. 2 as being the same type of LSTM, although each LSTM can be tailored to particular node types. The neighbor aggregator 210 aggregates encoded contents 212 from heterogeneous neighboring nodes for a particular node, by aggregating encoded contents from neighboring nodes that share a same node type, e.g., amongst the neighboring nodes. The neighbor aggregator 210 combines encoded contents 212 across neighboring nodes with different types, e.g., after combining encoded contents for neighboring nodes sharing a node type.

The LSTM 214 of the neighbor aggregator 210 is a network that is configured to optimize an objective function by maximizing the likelihood of preserving the overall heterogeneous graph structure. Although described as an optimization of generating node embeddings 150 that preserve heterogeneity in the input, e.g., graph 102, the objective function can also any type of optimization technique, e.g., minimizing a loss in feature data/size when encoding and aggregating node data. Each LSTM 214 can be trained separately to provide further granularity when preserving the heterogeneity of the graph 102 in the node embeddings 150.

The graph neural network 124 also includes an embedding fuser 220, which is configured to combine the node embeddings 150 into a smaller number of embeddings representing the graph 102, e.g., accessed or generated by the graph generator 162. In some implementations, the embedding fuser 220 generates a single embedding from the node embeddings 150. The graph neural network 124 includes a graph reconstructor 222 that generates a reconstructed graph based on the fused embedding(s) from the node embeddings 150, in which the graph reconstructor 222 determines a graph reconstruction loss. The graph reconstruction loss can be a value, e.g., computed by the graph reconstructor 222, that indicates how accurately the node embeddings 150 represent the features contained in the graph 102. In some implementations, the graph reconstruction loss can be an error value, in which the graph neural network 124 can be additionally trained to improve the quality of node embeddings 150 by minimizing graph reconstruction loss. In some implementations, the graph neural network 124 can be configured to generate node embeddings 150 until a threshold value for graph reconstruction is met. Further description of training the graph neural network is described in reference to FIG. 5 below.

FIG. 3 is a block diagram 300 that illustrates an example of the subgraph neural network 106 configured to generate node embeddings 152. The block diagram 300 illustrates the subgraph generator 166 that includes graph 102, in the form of subgraphs 146-1-146-N, each corresponding to a particular healthcare provider with associated patient nodes and patient health data nodes. The subgraph neural network 106 generates a respective subgraph embedding 152-1-152-N for each subgraph 146-1-146-N. e.g., corresponding to a healthcare provider. In other words, the subgraph neural network 106 generates the subgraph embeddings by determining topological properties of the respective subgraph.

The subgraph generator 166 provides subgraphs 146-1-146-N (also referred to as subgraph data 168 in FIG. 1B) to subgraph neural network 106. The subgraph neural network 106 includes subgraph analyzer 302, which is configured to analyze characteristics of each subgraph such as structure, neighborhood, and position. For example the subgraph analyzer 302 includes a structure analyzer 304 to analyze edges in the subgraph, a neighborhood analyzer 306 to analyze neighboring nodes in the subgraph, and a position analyzer 308 to compute distances between components of the subgraph. Collectively, the structure analyzer 304, the neighborhood analyzer 306, and the position analyzer 308 can be referred to as “module analyzers” for the subgraph neural network 106. In some implementations, processes performed by the module analyzers is performed concurrently, sequentially, etc.

The subgraph analyzer 302 identifies a set of internal nodes and set of border nodes for each subgraph 146-1-146-N. The set of internal nodes for a healthcare provider subgraph are patient nodes and patient health data nodes inside of the subgraph that are connected to two or more other nodes in the subgraph. As discussed in reference to FIG. 1B above, two or more nodes of a graph are connected by edges indicating a confirmed or possible co-appearance of the connected nodes. The border nodes for a healthcare provider subgraph includes nodes that are within a k-hop neighborhood of any node. Each border node is a node in the healthcare provider subgraph that is within a radial distance of any internal node in the subgraph. The border nodes can define a topological boundary that encloses the internal nodes of the subgraph. The subgraph analyzer 302 provides the set of internal nodes and the set of border nodes to each of the module analyzers, e.g., structure analyzer 304, neighborhood analyzer 306, and position analyzer 308.

The structure analyzer 304 analyzes the structure of each subgraph based on the set of internal nodes and the set of border nodes for the respective subgraph. For example, the structure analyzer 304 identifies edges connecting the internal nodes to neighboring border nodes for a respective subgraph. By identifying connecting edges in the subgraph, the structure analyzer 304 determines internal connectivity (e.g., connectivity of internal nodes in the subgraph) and border connectivity (e.g., connectivity of border nodes in the subgraph). The structure analyzer 304 generates a lower dimensional representation of the connectivity between nodes in the subgraph, while preserving properties of connectedness of nodes in a data structure. For example, the structure analyzer 304 can describe the connectivity of nodes through a data structure that describes adjacency in the subgraph, e.g., a matrix, a list, a set.

The neighborhood analyzer 306 analyzes the neighborhoods of each subgraph based on internal nodes and the set of border nodes for the respective subgraph. For example, the neighborhood analyzer 306 identifies sets of neighboring nodes within a number of k-hops of a particular node in a subgraph. The number of k-hops can be a radial distance (e.g., a number of traversals from one node to the next) from the particular node to identify a set of neighboring nodes of the particular node in the subgraph. By identifying sets of neighboring nodes from each node in the subgraph, the neighborhood analyzer 306 generates a data structure representing neighborhoods of nodes, e.g., clustering patient nodes and patient health data nodes with a degree of similarity between the nodes.

The position analyzer 308 analyzes the positions of each subgraph based on internal nodes and the set of border nodes for the respective subgraph, by computing distances between nodes in the subgraph. For example, the position analyzer 308 computes distances from nodes in the set of border nodes of the subgraph to neighboring nodes of the graph, e.g., a border distance from nodes in the set of border nodes to nodes in the subgraph that are adjacent to the set of the border nodes. The position analyzer 308 also computes a distance from a node in the set of internal nodes to other nodes of the subgraph, e.g., an internal distance of the subgraph.

In addition to each of the module analyzers determining characteristics of subgraphs in the graph, the subgraph analyzer 302 generates a set of anchor patches for each module analyzer based on the graph that contains subgraphs 146-1-146-N. Each anchor patch in a respective set of anchor patches of a module analyzer includes a random sample of nodes and edges of the graph. By randomly sampling nodes and edges of the graph, the subgraph analyzer 302 provides a randomly generated subgraph, e.g., an anchor patch, as an unbiased analysis of random neighborhoods in the graph to train the encoding process of subgraphs, e.g., generating the subgraph embeddings.

For each module analyzer, the subgraph analyzer 302 generates and provides neural network messages describing subgraph properties from the set of anchor patch to subgraph components, e.g., set of internal nodes, set of border nodes, edges. The neural network messages describe sampling, encoding, and estimation processes for generating subgraph embeddings, e.g., each subgraph embedding corresponding to a healthcare provider. For example, the neural network messages can describe characteristics of the anchor patch that are propagated to a subgraph for each module analyzer. By propagating neural network messages, properties of subgraph topology are encoded into a data structure that represents a healthcare provider. In some implementations, the subgraph analyzer 302 samples subgraph components (e.g., nodes, edges) of anchor patches based on the neural network messages. The subgraph analyzer 302 encodes properties of the subgraph components to a data structure with a lower dimension than the anchor patch. The encoding process can be performed repeatedly until a threshold value for similarity between an anchor patch and a subgraph is achieved. For example, a score indicating similarity between an anchor patch and a subgraph can be computed by the subgraph analyzer 302 based on subgraph characteristics, e.g., structure, neighborhood, position.

The subgraph neural network 106 includes a subgraph component readout 310, which can be an example neural network to output subgraph embeddings corresponding to healthcare providers, e.g., subgraph embeddings 152-1-152-N. For example, the subgraph component readout 310 compiles learned characteristics of data from subgraphs and anchor patches of the graph, e.g., as data structures with encoded data. The data structures are combined based on healthcare provider subgraphs 146-1-146-N to generate subgraph embeddings 152-1-152-N that includes relationships for healthcare providers that are identified and/or provided by other data sources.

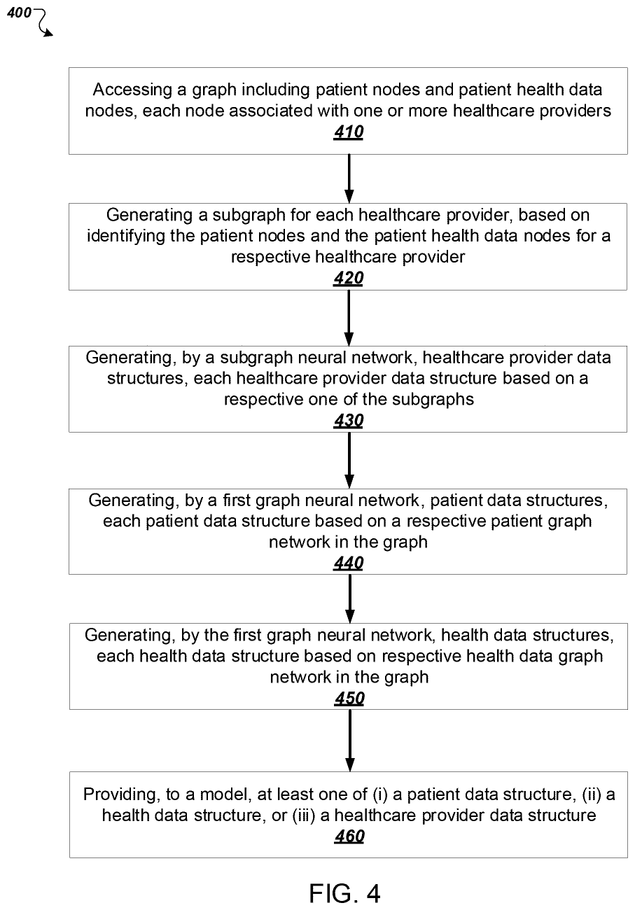

FIG. 4 is a flow diagram that illustrates an example of a process 400 for generating embeddings of the graph representing healthcare data from disparate data sources using the graph fusion system. The process 400 can be performed, for example, by the graph fusion system 132, which can include one or more computers, illustrated in FIGS. 1B and 1C.

The graph fusion system accesses a graph that includes nodes connected by edges (410). The nodes of the accessed graph are associated with one or more healthcare providers, e.g., medical professionals that can include but are not limited to nurses, physicians, hospital staff, and pharmacists. The nodes are categorized into subsets, including a set of patient nodes and a set of patient health data nodes. For example, each node in the set of patient nodes indicate a particular patient of a particular healthcare provider and each node in the set of patient health data nodes indicate a particular prescription, diagnosis, and/or symptom of a patient.

In some implementations, the graph fusion system receives at least some or all of the data related to healthcare providers, patients, and patient health data from data sources, and generate the graph to determine embeddings of the data. For example, the graph fusion system determines nodes and edges for the graph based on patient data and patient health data, e.g., received from data sources. In some implementations, portions of the graph such as subgraphs, nodes, and edges, are updated and/or generated, e.g., in response to receiving additional data from data sources.

The graph fusion system generates subgraphs, by identifying patient nodes and patient health data nodes for each healthcare provider (420). The graph fusion system identifies patient nodes representing patients and patient health data nodes for the patients, e.g., indicated by edges that connect nodes in the accessed graph, for each healthcare provider. Each subgraph includes the identified nodes for the respective healthcare provider and also includes the edges between nodes, e.g., indicating patient health data for the patients of the patient nodes in the subgraph.

The graph fusion system generates, by a subgraph neural network, healthcare provider data structures for each subgraph (430). The healthcare provider data structures have a lower dimension than the corresponding subgraph, e.g., as an embedding that is a dense representation of the feature data in the subgraph. The feature data of the subgraph can include patient measurements, health records, lab reports, diagnostics, etc. performed by the healthcare provider. The healthcare provider data structure for a generated subgraph is an embedding of the corresponding subgraph, e.g., the embedding is a tensor-based representation of the features in the subgraph with multi-dimensional relationships of the features. In some implementations, the embedding is a vector-based representation of the features in the subgraph, in which the vector-based embedding describes one-dimensional relationships of the features.

In some implementations, generating a healthcare provider data structure for a subgraph includes determining a set of internal nodes and a set of border nodes within the subgraph. Each internal node of the subgraph is connected by edges to two or more nodes in the subgraph and each border node corresponding to a node of the subgraph within a radial distance of the internal nodes within the subgraph. A set of characteristics is be defined for the subgraph of the healthcare provider data structure based on the set of internal nodes and the set of border nodes. A set of anchor patches for the accessed graph is generated by randomly sampling different portions of the graph, e.g., a random sample of the nodes and edges of the graph. Based on the set of the anchor patches, one or more messages are transmitted (e.g., propagated through layers of the subgraph neural network 106) from the set of anchor patches to the set of anchor nodes, the set of border nodes, and the edges of the subgraph. The messages encode properties of at least one anchor patch onto the set of internal nodes, the set of border nodes, and/or the edges of the subgraph. The healthcare data structure for the subgraph are generated based on the set of the characteristics and the messages for the subgraph.

In some implementations, defining a set of characteristics for the subgraph when generating the healthcare provider data structure for the subgraph includes computing a border distance from each node in the set of border nodes of the subgraph to neighboring nodes of the graph. The subgraph does not include neighboring nodes of the graph and the neighboring nodes are adjacent to at least one node in the set of border nodes. An internal distance from a respective node in the subgraph to other nodes in the subgraph is computed for each node in the subgraph. A set of subgraph neighboring nodes is identified for each node in the subgraph, and the set of subgraph neighboring nodes for a respective node includes nodes of the subgraph within a radial distance of the respective node. Based on the set of subgraph neighboring nodes for each node in the subgraph, the structure of the subgraph is determined based on a degree of connectivity for nodes and/or edges of the subgraph connecting to the set of internal nodes of the subgraph. In some examples, the radial distance is a hyperparameter that configures the training of the subgraph neural network, e.g., a number of k-hops to be performed.

In some implementations generating the particular healthcare provider data structure includes sampling the random sample of nodes and edges from the set of anchor patches based on the one or more messages for the subgraph. Each anchor patch in the set of anchor patches is encoded to a lower dimension than a dimension of the corresponding anchor patch. A similarity between each anchor patch in the set of anchor patches and the respective subgraph is estimated using the set of characteristics for the subgraph

The graph fusion system generates, by a first graph neural network, patient data structures for patient graph networks in the accessed graph (440). Each patient graph network represents a particular patient, e.g., a particular patient node, and the patient health data for the particular patient, e.g., patient health data nodes that are connected to the particular patient node. In some implementations, the graph fusion system identifies particular patient health data nodes that are connected to the patient node, for each patient node in the accessed graph. The graph fusion system generates the patient graph networks for each patient represented in the accessed graph, e.g., as subgraphs that include the patient node of the patient and edges connecting to other nodes representing health data for the patient.

As an example, the patient graph network includes a patient node with multiple patient health data nodes with at least one healthcare provider, e.g., including multiple types and instances of different healthcare providers. The patient data structure is a lower dimensional representation of the patient graph network, e.g., an embedding that is a dense representation of the feature data in the patient graph network. The feature data of the patient graph network can include patient measurements, health records, lab reports, diagnostics, etc. performed by one or more healthcare providers of the patient. Similarly to the healthcare provider data structure discussed above, the patient data structure can be a tensor-based or vector-based representation of the features for a patient graph network.

The graph fusion system generates, by the first graph neural network, health data structures for health data graph networks in the accessed graph (450). Each health data graph network in the accessed graph includes a particular patient health data node and some or all of the patient nodes connected to the particular patient health data node by edges. The health data structure is an embedding of the health data graph network, e.g., a lower dimensional representation of the features of the health data in the health data graph network.

The graph fusion system provides at least one of (i) a patient data structure, (ii) a health data structure, or (iii) a healthcare provider data structure, to a model (460). By providing lower dimensional representations of feature data from multiple data sources, including disparate sources with different types, formats, etc., the graph fusion system provides a standardized data input for further application. For example, predictive models can leverage the embeddings for patient, patient health data, and/or healthcare providers to more efficiently predict trends in the utilization of some types of drugs over others for particular symptoms, e.g., compared to predictive models that process data from disparate sources. The graph fusion system can enable improvements in computing systems and devices by providing embeddings that can be trained, e.g., improving identification of relationships or patterns between two or more seemingly unrelated data sources. For example, an embedding can more accurately capture a trend in prescription of a particular drug among healthcare providers based on data from healthcare organizers and drug marketing materials, e.g., a drug that is more well-understood by healthcare providers for treating a particular symptom can be prescribed at a higher rate than a drug that is unfamiliar to healthcare providers.

In some implementations, graph fusion system is configured to generate a second graph representing a healthcare provider network based on the healthcare provider data structures and external healthcare data. The second graph includes healthcare provider nodes that each represent a respective healthcare provider, in which the healthcare provider nodes are connected by edges described by the external healthcare data sources, e.g., healthcare organizations, payer access, physician early adoption, and/or healthcare provider marketing.

In some implementations, the first graph neural network generates the patient data structure for a particular patient node, by sampling neighboring nodes of the particular patient node and encoding data from the neighboring nodes. The data from neighboring nodes is encoded, e.g., from a first format provided by the data source of the node to a second format for the patient data structure, in which the second format is a lower dimension than the first format and represents the features of the data using a format of numbers, characters, values, etc. in a vector or tensor. In some implementations, encoding the data from neighboring nodes is performed by pre-training the first graph neural network to generate patient data structures using the data from the neighboring nodes and aggregating the data based on the type of node, e.g., patient node, patient health data node. Aggregating data for a particular node can include aggregating data from neighboring nodes that share a node type as the particular node, and combining data from neighboring nodes having a different node type than the node type of the particular node.

In some implementations, sampling the neighboring nodes of the particular patient node includes performing a random walk with restart from the particular patient node to at least one of the neighboring nodes, selecting a number of top random walks for each type of node in the neighboring nodes, and performing the number of top random walks using the neighboring nodes.

In some implementations, the selection of the number of top random walks is based on the complexity of the accessed graph, e.g., related to the density of nodes and edges of the accessed graph. For example, a large number (e.g., ten or more) of random walks is performed when sampling neighboring nodes in a dense graph compared to a small number (e.g., five or fewer) of random walks when sampling neighboring nodes in a sparse graph.

In some implementations, generating the patient data structure for the particular patient node further includes aggregating the data from the one or more neighboring nodes to the patient data structure for the particular patient node and generating an objective function for the patient data structure. The objective function for generating the patient data structure indicates a likelihood of preserving the patient graph network of the graph, based on the patient data structure.

In some implementations, the first graph neural network is a heterogeneous neural network further includes a long short-term memory network. The long short-term memory network can be configured to aggregate data from the nodes of the patient graph network based on an order of the nodes, by aggregating the data based on a chronological order for which the node was generated, accessed, etc. In some implementations, networks and models such as transformers, recurrent neural networks, and other types of memory models are leveraged and trained to aggregate data from nodes.

In some implementations, the second graph neural network for generating external healthcare data structures are the same neural network as the first graph neural network that generates health data structures. For example, leveraging learned relationships from training the first graph neural network and generating embeddings of subgraphs from the first graph neural network can further improve training and embedding generation through the second graph neural network, and vice-versa. In some implementations, the second graph neural network is a heterogeneous neural network, similarly described above in reference to the first graph neural network in step 430 above.

FIG. 5 is a flow diagram that illustrates an example of a process for training the graph neural network of the graph fusion system to generate node embeddings. The process 500 can be performed by computing devices and/or systems, such as the graph fusion system 132 illustrated in FIGS. 1B and 1C. The graph neural network can be the first graph neural network and/or the second graph neural network, described in reference to process 400 above.

Training the graph neural network includes generating the patient data structures and the health data structures based on respective graph networks from the accessed graph (510). For example, embeddings such as patient data structures are generated for patient graph networks for a particular patient node in the accessed graph, e.g., as a subgraph that includes the particular patient node of the patient and edges connecting to patient health data nodes from the particular patient node. The patient health data nodes include patient health data, e.g., measurements, prescriptions, diagnoses, symptoms, for the particular patient. As another example, embeddings such as health data structures are generated for the health data graph network, e.g., a subgraph of the accessed graph that includes a particular patient health data node, and some or all of the patient nodes connected to the particular patient health data node by edges.

Training the graph neural network includes combining the patient data structures and the health data structures to generate a reconstructed graph (520). The reconstructed graph is generated by combining all of the patient data structures and the health data structures to generate a full set of data structures, e.g., a full set of embeddings to represent a reconstruction of the accessed graph. A computing device, including the graph fusion system 132, can be used to process the full set of data structures to determine nodes and edges on the reconstructed graph. In some implementations, the reconstructed graph is an embedding itself, e.g., to be compared to another embedding of the accessed graph.

Training the graph neural network includes comparing the reconstructed graph based on the combined patient data structures and health data structures to the accessed graph (530). For example, a number of characteristics of the reconstructed graph and the accessed graph are compared, including structural similarity, degrees of each node, network distances between nodes, etc. In some implementations, an embedding of the reconstructed graph and an embedding of the accessed graph are compared, including comparing vectors by computing a difference, distance, etc.

Based on the comparison of the reconstructed graph and the accessed graph, training the graph neural network includes updating one or more parameters of the first graph neural network (540). For example, a difference between the reconstructed graph and the accessed graph is computed. Upon determining that the difference between the graphs exceeds a threshold value, e.g., heuristically determined, a hyperparameter, the graph fusion system updates parameters of the graph neural network. In more detail, the first graph neural network can include one or more fully or partially connected layers. Each of the layers include one or more parameter values indicating an output of the layers. In some implementations, parameter adjustment includes adjusting weights of the neural network layers. The layers of the model can generate embeddings of data nodes and edges from disparate data sources, which can be used as an input for predictive models.

FIG. 6 is a flow diagram that illustrates an example of a process for training the subgraph neural network of the graph fusion system to generate subgraph embeddings. The process 600 can be performed by computing devices and/or systems, for example, the graph fusion system 132 illustrated in FIGS. 1B and 1C. The subgraph neural network is configured to generate healthcare provider data structures, described in reference to process 600 above.

Training the subgraph neural network includes generating the healthcare provider data structures based on respective subgraphs from the accessed graph (610). For example, embeddings such as healthcare provider data structures are generated for subgraphs, each subgraph including identified patient nodes and patient health data nodes for a healthcare provider. The healthcare provider data structures represent an embedding with a lower dimension than the corresponding subgraph, e.g., as an embedding that is a dense representation of the feature data in the subgraph.

Training the graph neural network includes combining the healthcare provider data structures to generate a reconstructed graph (620). The reconstructed graph is generated by combining all of the healthcare provider data structures to generate a set of data structures, e.g., a set of embeddings to represent a reconstruction of the accessed graph. A computing device, including the graph fusion system 132, processes the set of data structures to determine nodes and edges on the reconstructed graph. In some implementations, the reconstructed graph is an embedding itself, e.g., to be compared to another embedding of the accessed graph.

Training the graph neural network includes comparing the reconstructed graph based on the combined healthcare provider data structures to the accessed graph (630). For example, a number of characteristics of the reconstructed graph and the accessed graph are compared, including structural similarity, degrees of each node, network distances between nodes, etc. In some implementations, an embedding of the reconstructed graph and an embedding of the accessed graph are compared, including comparing vectors by computing a difference, distance, etc. In some implementations, the healthcare provider data structures are compared to respective subgraphs of the accessed graph that is being embedded, e.g., representing the features of the subgraph in a lower dimension.

Based on the comparison of the reconstructed graph and the accessed graph, training the subgraph neural network includes updating one or more parameters of the subgraph neural network (640). For example, a difference between the reconstructed graph and the accessed graph can be computed. Upon determining that the difference between the graphs exceed a threshold value, e.g., heuristically determined, a hyperparameter, the graph fusion system 132 updates parameters of the subgraph neural network. As described above in reference to process 600, the subgraph neural network 106 also includes one or more fully or partially connected layers, each layer can include one or more parameter values indicating an output of the layers. These parameters can include weights of neural network layers in the subgraph neural network 106 to be adjusted, in which the subgraph neural network can be continually updated until a value is met, e.g., below a threshold error.

For example, the graph fusion system 132 (referring to FIG. 1C above) can receive a baseline model output from a model configured to perform a downstream task, e.g., using data analytics, predictive modeling, or other techniques. The model generates a baseline model output generated by performing the downstream task absent embeddings of nodes and subgraphs. The model generates an updated model output using an embedding generated by the graph fusion system 132, such as patient data structures, health data structures, healthcare provider data structures, or some combination herein. The graph fusion system 132 compares the updated model output to the baseline model output and adjusts parameters, hyperparameters, etc. of the graph neural network 116, 124, and/or subgraph neural network 106. In some implementations, the graph fusion system 132 can generate updated embeddings based on the newly generated model output.

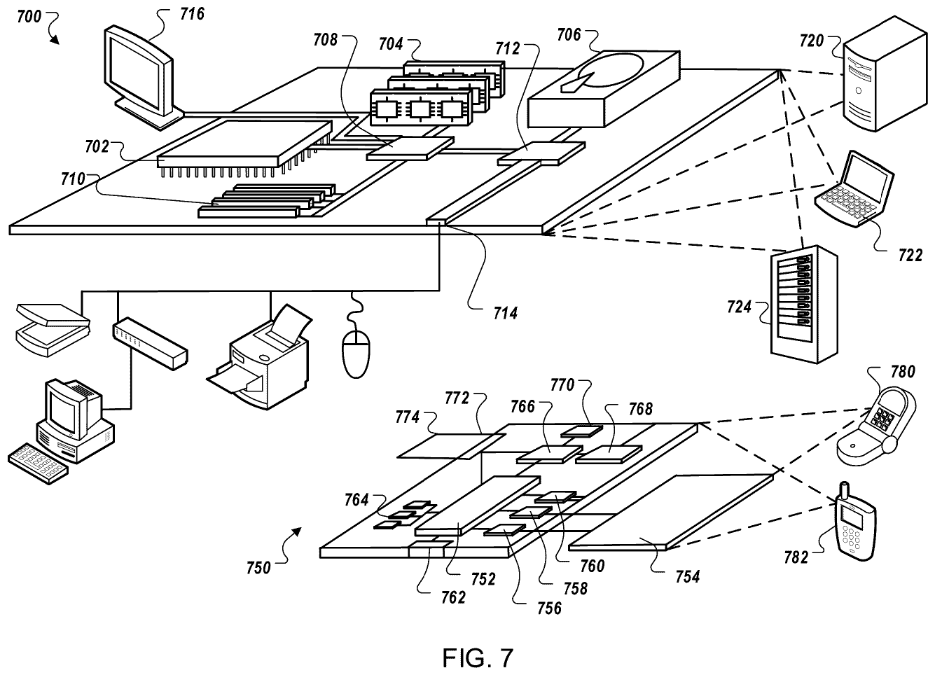

FIG. 7 is a block diagram of computing devices 700, 750 that may be used to implement the systems and methods described in this document, as either a client or as a server or multiple servers. As an example, computing device 131 can be an example of computing devices 700, 750 to generate node and subgraph embeddings. Computing device 700 and 750 is intended to represent various forms of digital computers, such as laptops, desktops, workstations, personal digital assistants, servers, blade servers, mainframes, and other appropriate computers. The components shown here, their connections and relationships, and their functions, are meant to be exemplary only, and are not meant to limit implementations described and/or claimed in this document.

Computing device 700 includes a processor 702, memory 704, a storage device 706, a high-speed interface 708 connecting to memory 704 and high-speed expansion ports 710, and a low speed interface 712 connecting to low speed bus 714 and storage device 706. Each of the components 702, 704, 706, 708, 710, and 712, are interconnected using various busses, and may be mounted on a common motherboard or in other manners as appropriate. The processor 702 can process instructions for execution within the computing device 700, including instructions stored in the memory 704 or on the storage device 706 to display graphical information for a GUI on an external input/output device, such as display 716 coupled to high speed interface 708. In other implementations, multiple processors and/or multiple buses may be used, as appropriate, along with multiple memories and types of memory. Also, multiple computing devices 700 may be connected, with each device providing portions of the necessary operations, e.g., as a server bank, a group of blade servers, or a multi-processor system.

The memory 704 stores information within the computing device 700. In one implementation, the memory 704 is a computer-readable medium. In one implementation, the memory 704 is a volatile memory unit or units. In another implementation, the memory 704 is a non-volatile memory unit or units.

The storage device 706 is capable of providing mass storage for the computing device 700. In one implementation, the storage device 706 is a computer-readable medium. In various different implementations, the storage device 706 may be a floppy disk device, a hard disk device, an optical disk device, or a tape device, a flash memory or other similar solid-state memory device, or an array of devices, including devices in a storage area network or other configurations. In one implementation, a computer program product is tangibly embodied in an information carrier. The computer program product contains instructions that, when executed, perform one or more methods, such as those described above. The information carrier is a computer- or machine-readable medium, such as the memory 704, the storage device 706, or memory on processor 702.

The high-speed controller 708 manages bandwidth-intensive operations for the computing device 700, while the low speed controller 712 manages lower bandwidth-intensive operations. Such allocation of duties is exemplary only. In one implementation, the high-speed controller 708 is coupled to memory 704, display 716, e.g., through a graphics processor or accelerator, and to high-speed expansion ports 710, which may accept various expansion cards (not shown). In the implementation, low-speed controller 712 is coupled to storage device 706 and low-speed expansion port 714. The low-speed expansion port, which may include various communication ports, e.g., USB, Bluetooth, Ethernet, wireless Ethernet, may be coupled to one or more input/output devices, such as a keyboard, a pointing device, a scanner, or a networking device such as a switch or router, e.g., through a network adapter.

The computing device 700 may be implemented in a number of different forms, as shown in the figure. For example, it may be implemented as a standard server 720, or multiple times in a group of such servers. It may also be implemented as part of a rack server system 724. In addition, it may be implemented in a personal computer such as a laptop computer 722. Alternatively, components from computing device 700 may be combined with other components in a mobile device (not shown), such as device 750. Each of such devices may contain one or more of computing device 700, 750, and an entire system may be made up of multiple computing devices 700, 750 communicating with each other.

Computing device 750 includes a processor 752, memory 764, an input/output device such as a display 754, a communication interface 766, and a transceiver 768, among other components. The device 750 may also be provided with a storage device, such as a microdrive or other device, to provide additional storage. Each of the components 750, 752, 764, 754, 766, and 768, are interconnected using various buses, and several of the components may be mounted on a common motherboard or in other manners as appropriate.

The processor 752 can process instructions for execution within the computing device 750, including instructions stored in the memory 764. The processor may also include separate analog and digital processors. The processor may provide, for example, for coordination of the other components of the device 750, such as control of user interfaces, applications run by device 750, and wireless communication by device 750.

Processor 752 may communicate with a user through control interface 758 and display interface 756 coupled to a display 754. The display 754 may be, for example, a TFT LCD display or an OLED display, or other appropriate display technology. The display interface 756 may include appropriate circuitry for driving the display 754 to present graphical and other information to a user. The control interface 758 may receive commands from a user and convert them for submission to the processor 752. In addition, an external interface 762 may be provided in communication with processor 752, so as to enable near area communication of device 750 with other devices. External interface 762 may provide, for example, for wired communication, e.g., via a docking procedure, or for wireless communication, e.g., via Bluetooth or other such technologies.

The memory 764 stores information within the computing device 750. In one implementation, the memory 764 is a computer-readable medium. In one implementation, the memory 764 is a volatile memory unit or units. In another implementation, the memory 764 is a non-volatile memory unit or units. Expansion memory 774 may also be provided and connected to device 750 through expansion interface 772, which may include, for example, a SIMM card interface. Such expansion memory 774 may provide extra storage space for device 750, or may also store applications or other information for device 750. Specifically, expansion memory 774 may include instructions to carry out or supplement the processes described above, and may include secure information also. Thus, for example, expansion memory 774 may be provided as a security module for device 750, and may be programmed with instructions that permit secure use of device 750. In addition, secure applications may be provided via the SIMM cards, along with additional information, such as placing identifying information on the SIMM card in a non-hackable manner.

The memory may include for example, flash memory and/or MRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 764, expansion memory 774, or memory on processor 752.

Device 750 may communicate wirelessly through communication interface 766, which may include digital signal processing circuitry where necessary. Communication interface 766 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 768. In addition, short-range communication may occur, such as using a Bluetooth, WiFi, or other such transceiver (not shown). In addition, GPS receiver module 770 may provide additional wireless data to device 750, which may be used as appropriate by applications running on device 750.

Device 750 may also communicate audibly using audio codec 760, which may receive spoken information from a user and convert it to usable digital information. Audio codec 760 may likewise generate audible sound for a user, such as through a speaker, e.g., in a handset of device 750. Such sound may include sound from voice telephone calls, may include recorded sound, e.g., voice messages, music files, etc., and may also include sound generated by applications operating on device 750.

The computing device 750 may be implemented in a number of different forms, as shown in the figure. For example, it may be implemented as a cellular telephone 780. It may also be implemented as part of a smartphone 782, personal digital assistant, or other similar mobile device.

Various implementations of the systems and techniques described here can be realized in digital electronic circuitry, integrated circuitry, specially designed ASICs, computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs, also known as programs, software, software applications or code, include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device, e.g., magnetic discs, optical disks, memory, Programmable Logic Devices (PLDs) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The systems and techniques described here can be implemented in a computing system that includes a back-end component, e.g., as a data server, or that includes a middleware component such as an application server, or that includes a front-end component such as a client computer having a graphical user interface or a Web browser through which a user can interact with an implementation of the systems and techniques described here, or any combination of such back-end, middleware, or front-end components. The components of the system can be interconnected by any form or medium of digital data communication such as, a communication network. Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

In addition, certain data may be treated in one or more ways before it is stored or used, so that personally identifiable information is removed. For example, in some embodiments, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over what information is collected about the user, how that information is used, and what information is provided to the user.

A number of embodiments have been described. Nevertheless, it will be understood that various modifications may be made without departing from the spirit and scope of the invention. Accordingly, other embodiments are within the scope of the following claims. While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment.

Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a sub combination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, some processes depicted in the accompanying figures do not necessarily require the particular order shown, or sequential order, to achieve desirable results.

1. A computer-implemented method performed by one or more computers comprising:
accessing a graph comprising nodes connected by edges, wherein:
a first subset of the nodes are patient nodes each representing a corresponding patient, wherein each patient is associated with one or more healthcare providers, and
a second subset of the nodes are patient health data nodes each representing health data of a corresponding patient, wherein the health data of each patient health data node is associated with at least one of the one or more healthcare providers;
generating one or more subgraphs based on the graph, comprising, for each subgraph:
identifying the patient nodes and the patient health data nodes in the graph that are associated with a particular one of the healthcare providers, and generating the subgraph comprising the identified nodes;
generating, by a subgraph neural network, healthcare provider data structures, each healthcare provider data structure based on a respective one of the subgraphs, wherein each healthcare provider data structure has a lower dimension than the corresponding subgraph;
generating, by a first graph neural network, patient data structures, each patient data structure based on a respective patient graph network in the graph, each patient graph network comprising a particular one of the patient nodes of the graph and the patient health care data nodes connected to the particular patient node by edges, wherein each patient data structure has a lower dimension than the corresponding patient graph network;
generating, by the first graph neural network, health data structures, each health data structure based on a respective health data graph network in the graph, each health data graph network comprising a particular one of the patient health data nodes of the graph and the patient nodes connected to the particular patient health data node by edges, wherein each health data structure has a lower dimension than the corresponding health data graph network; and
providing to a model, at least one of (i) a patient data structure from the patient data structures or (ii) a health data structure from the health data structures or (iii) a healthcare provider data structure from the healthcare provider data structures.

2. The computer-implemented method of claim 1, comprising:
receiving patient data and patient health data from one or more data sources;
determining nodes and edges for the graph based on the patient data and the patient health data; and
generating at least a portion of the graph based on the determined nodes and edges.

3. The computer-implemented method of claim 1, wherein generating the patient data structures comprises, for each patient data structure:
sampling, for the particular patient node of the patient data structure, one or more neighboring nodes of the particular patient node; and
encoding, for the particular patient node, data from the one or more neighboring nodes to generate the patient data structure for the particular patient node.

4. The computer-implemented method of claim 3, comprising:
aggregating the data from the one or more neighboring nodes to the patient data structure for the particular patient node; and
generating an objective function for the patient data structure, the objective function indicative of a likelihood of preserving the patient graph network of the graph based on the patient data structure.

5. The computer-implemented method of claim 3, wherein sampling the one or more neighboring nodes of the particular patient node comprises:
performing a random walk with restart from the particular patient node, wherein performing the random walk with restart comprises traversing the graph from the particular patient node to a selected node in the one or more neighboring nodes, wherein the selected node is selected based on a probabilistic distribution that is sampled when the random walk is performed from the particular patient node;
selecting, for each type of node in the one or more neighboring nodes, a number of top random walks from the particular patient node, wherein the type of a node indicates whether the node is a patient node or a patient health data node; and
performing the number of top random walks using the one or more neighboring nodes of the particular patient node.

6. The computer-implemented method of claim 5, wherein selecting the number of top random walks for each node in the graph is based on a density of the graph, the density of the graph describing a ratio of edges in the graph and a maximum number of edges contained in the graph.

7. The computer-implemented method of claim 3, wherein encoding the data from the one or more neighboring nodes of the particular patient node comprises:
pre-training the first graph neural network to generate the patient data structures based on the data from the one or more neighboring nodes; and
aggregating the data from the one or more neighboring nodes of the respective patient graph networks based on a type of node, wherein the type of a node indicates whether the node is a patient node or a patient health data node.

8. The computer-implemented method of claim 3, in which the first graph neural network can be trained by performing operations comprising:
generating the patient data structures and the health data structures based on respective graph networks from the accessed graph;
combining the patient data structures and the health data structures to generate a reconstructed graph;
comparing the reconstructed graph based on the combined patient data structures and health data structures to the accessed graph; and
based on the comparison of the reconstructed graph and the accessed graph, updating one or more parameters of the first graph neural network.

9. The computer-implemented method of claim 3, in which the subgraph neural network can be trained by performing operations comprising:
generating the healthcare provider data structures based on the respective subgraphs from the accessed graph;
combining the healthcare provider data structures to generate a reconstructed graph;
comparing the reconstructed graph based on the healthcare provider data structures to the accessed graph; and
based on the comparison of the reconstructed graph and the accessed graph, updating one or more parameters of the subgraph neural network.

10. The computer-implemented method of claim 4, wherein aggregating the data from the one or more neighboring nodes to the patient data structure for the particular patient node comprises:
aggregating the data from the one or more neighboring nodes with a same node type as the particular patient node, the type of a node indicates whether the node is a patient node or a patient health data node; and
combining data from at least one of the neighboring nodes, the at least one of the neighboring nodes having a different type than a type of the particular patient node.

11. The computer-implemented method of claim 1, wherein the first graph neural network is a heterogeneous neural network that comprises a long short-term memory network configured to aggregate data from the nodes based on an order of the nodes.

12. The computer-implemented method of claim 1, wherein the health data of a patient comprises at least one of (i) a diagnosis, (ii) a prescription, or (iii) a symptom, for the patient.

13. The computer-implemented method of claim 1, comprising generating a second graph representing a healthcare provider network based on the healthcare provider data structures and external healthcare data, the second graph comprising healthcare provider nodes each representing a respective healthcare provider, the healthcare provider nodes connected by edges.

14. The computer-implemented method of claim 13, wherein the external healthcare data comprise data related to one or more of (i) healthcare organizations, (ii) payer access, (iii) physician early adoption, or (iv) healthcare provider marketing.

15. The computer-implemented method of claim 14, comprising:
generating, by a second graph neural network, second healthcare provider data structures, each second healthcare provider data structure based on a respective healthcare provider graph network in the second graph, each healthcare provider graph network comprising a particular one of the healthcare provider nodes of the second graph and nodes connected to the particular healthcare provider node by edges, wherein each second healthcare provider data structure has a lower dimension than the corresponding healthcare provider graph network.

16. The computer-implemented method of claim 14, comprising:
generating, by a second graph neural network, external healthcare data structures, each external healthcare data structure based on a respective healthcare provider graph network in the second graph and the external healthcare data, each healthcare provider graph network comprising a particular one of the healthcare provider nodes of the second graph and nodes connected to the particular healthcare provider node by edges, wherein each external healthcare data structure has a lower dimension than the corresponding external healthcare data.

17. The computer-implemented method of claim 14, wherein the first graph neural network and the second graph neural network are the same neural network.

18. The computer-implemented method of claim 1, wherein generating a particular healthcare provider data structure comprises:
determining a set of internal nodes and a set of border nodes within the subgraph for the health care provider data structure, each internal node connected by edges to two or more nodes in the subgraph and each border node corresponding to a node of the subgraph within a radial distance of the internal nodes within the subgraph;
defining a set of characteristics for each subgraph for the health care provider data structure based on the set of internal nodes and the set of border nodes;
generating a set of anchor patches the graph, each anchor patch representing a random sample of the nodes and the edges of the graph;
providing one or more messages from the set of anchor patches to the set of internal nodes, the set of border nodes, and the edges of the subgraph, the one or more messages encoding properties of at least one anchor patch from the set of anchor patches onto at least one of (i) the set of internal nodes, (ii) the set of border nodes, or (iii) the edges, of the subgraph; and
generating, based on the set of characteristics and the one or more messages for the subgraph, the particular healthcare provider data structure.

19. The computer-implemented method of claim 18, wherein defining the set of characteristics for a particular subgraph comprises:
computing a border distance from each node in the set of border nodes of the subgraph to neighboring nodes of the graph, wherein the subgraph does not include neighboring nodes of the graph and the neighboring nodes are adjacent to at least one node in the set of border nodes;
computing, for each node in the subgraph, an internal distances from a respective node in the subgraph to other nodes in the subgraph;
identifying, for each node in the subgraph, a set of subgraph neighboring nodes, wherein the set of subgraph neighboring nodes for a respective node comprises nodes of the subgraph within a radial distance of the respective node; and
determining a structure of the subgraph based on the set of subgraph neighboring nodes for each node in the subgraph, the structure of the subgraph comprising a degree of connectivity for at least one (i) the nodes in the subgraph, or (ii) the edges of the subgraph connecting to the set of internal nodes of the subgraph.

20. The computer-implemented method of claim 18, wherein generating the particular healthcare provider data structure comprises:
sampling the random sample of nodes and edges from the set of anchor patches based on the one or more messages for the subgraph;
encoding each anchor patch in the set of anchor patches to a lower dimension than a dimension of the corresponding anchor patch; and
estimating a similarity of each anchor patch in the set of anchor patches to the respective subgraph based on the set of characteristics for subgraph.

21. A graph fusion system comprising:
a computing device comprising at least one processor; and
a memory communicatively coupled to the at least one processor, the memory storing instructions which, when executed by the at least one processor, cause the at least one processor to perform operations comprising:
accessing a graph comprising nodes connected by edges, wherein:
a first subset of the nodes are patient nodes each representing a corresponding patient, wherein each patient is associated with one or more healthcare providers, and
a second subset of the nodes are patient health data nodes each representing health data of a corresponding patient, wherein the health data of each patient health data node is associated with at least one of the one or more healthcare providers;
generating one or more subgraphs based on the graph, comprising, for each subgraph:
identifying the patient nodes and the patient health data nodes in the graph that are associated with a particular one of the healthcare providers, and
generating the subgraph comprising the identified nodes;
generating, by a subgraph neural network, healthcare provider data structures, each healthcare provider data structure based on a respective one of the subgraphs, wherein each healthcare provider data structure has a lower dimension than the corresponding subgraph;
generating, by a first graph neural network, patient data structures, each patient data structure based on a respective patient graph network in the graph, each patient graph network comprising a particular one of the patient nodes of the graph and the patient health care data nodes connected to the particular patient node by edges, wherein each patient data structure has a lower dimension than the corresponding patient graph network;
generating, by the first graph neural network, health data structures, each health data structure based on a respective health data graph network in the graph, each health data graph network comprising a particular one of the patient health data nodes of the graph and the patient nodes connected to the particular patient health data node by edges, wherein each health data structure has a lower dimension than the corresponding health data graph network; and
providing by the graph fusion system to a model, at least one of (i) a patient data structure from the patient data structures, (ii) a health data structure from the health data structures, or (iii) a healthcare provider data structure from the healthcare provider data structures.

22. The graph fusion system of claim 21, further comprising:
receiving a model output from the model, the model configured to perform a downstream task to generate the model output using the at least one of (i) a patient data structure, (ii) a health data structure, or (iii) a healthcare provider data structure;
receiving a baseline model output from the model, the baseline model output generated by the model performing the downstream task absent the at least one of (i) a patient data structure, (ii) a health data structure, or (iii) a healthcare provider data structure;
comparing the model output to the baseline model output; and
updating, based on the comparison of the model output to the baseline model output, one or more parameters of at least one of (i) the first graph neural network, (ii) the second graph neural network, or (iii) the subgraph neural network, of the graph fusion system.

23. A non-transitory computer-readable storage device storing instructions that when executed by one or more processors of a computing device cause the one or more processors to perform operations comprising:
accessing a graph comprising nodes connected by edges, wherein:
a first subset of the nodes are patient nodes each representing a corresponding patient, wherein each patient is associated with one or more healthcare providers, and
a second subset of the nodes are patient health data nodes each representing health data of a corresponding patient, wherein the health data of each patient health data node is associated with at least one of the one or more healthcare providers;
generating one or more subgraphs based on the graph, comprising, for each subgraph:
identifying the patient nodes and the patient health data nodes in the graph that are associated with a particular one of the healthcare providers, and
generating the subgraph comprising the identified nodes;
generating, by a subgraph neural network, healthcare provider data structures, each healthcare provider data structure based on a respective one of the subgraphs, wherein each healthcare provider data structure has a lower dimension than the corresponding subgraph;
generating, by a first graph neural network, patient data structures, each patient data structure based on a respective patient graph network in the graph, each patient graph network comprising a particular one of the patient nodes of the graph and the patient health care data nodes connected to the particular patient node by edges, wherein each patient data structure has a lower dimension than the corresponding patient graph network;
generating, by the first graph neural network, health data structures, each health data structure based on a respective health data graph network in the graph, each health data graph network comprising a particular one of the patient health data nodes of the graph and the patient nodes connected to the particular patient health data node by edges, wherein each health data structure has a lower dimension than the corresponding health data graph network; and
providing to a model, at least one of (i) a patient data structure from the patient data structures or (ii) a health data structure from the health data structures or (iii) a healthcare provider data structure from the healthcare provider data structures.
